(19) United States
(12) Patent Application Publication
(10) Pub. No.: US 2005/0095269 A1
(43) Pub. Date: May 5, 2005

(54) Gel plug for blockage of the canaliculus

(76) Inventors: Parviz Robert Ainpour, Seal Beach, CA (US); James M. Christensen, Glendora, CA (US)

Correspondence Address: KOPPEL, JACOBS, PATRICK & HEYBL, 555 ST. CHARLES DRIVE, SUITE 107, THOUSAND OAKS, CA, 91360, US

(21) Appl. No.: 10/700,699

(22) Filed: Nov. 4, 2003

(51) Int. Cl. A61F 2/00; A61M 31/00

(52) U.S. Cl. 424/427; 604/500

(57) ABSTRACT

A cylindrical plug for insertion through the punctum and into the canaliculus to block the flow of tears therethrough comprises an insert of a gelatinous material which when hydrated expands and changes shape, conforming at least to a portion of the canaliculus crossection and contacting the walls of the canaliculus, thus blocking flow. The gel plug can be inserted in a dry state and hydrating in situ or hydrated, either partially or fully, prior to insertion.

### FIELD OF THE INVENTION

This invention relates generally to a reversible, high water content gel plug used to block fluid flow through the canalicular canal of the eye for the treatment of keratoconjunctivitis sicca (dry eye). More specifically, the present invention relates to a dry hydrogel rod, which swells to form a high water content gel that adapts to the size and shape of the patient's canaliculus, thereby occluding the channel. Hydration of the dry hydrogel rod may be accomplished prior to or after insertion, or partial hydration can occur prior to insertion followed by full hydration after insertion.

### BACKGROUND

Conditions of keratoconjunctivitis sicca (dry eye) have been treated with success by blocking the tear flow from the eye through the canaliculus. This has been accomplished by closing the canalicular canal by stitching the punctal opening shut or by using electrical or laser cauterization to seal the punctal opening or close the canalicular canal. Although such methodology can provide the desired results, the procedure is not reversible without reconstructive surgery. Since it is sometimes difficult to determine if a particular patient has a problem with excessive drainage or the tear production is too small, irreversible blockage is not a preferred approach.

One means of temporarily blocking the canaliculus for the treatment of dry eye is through the use of intracanalicular gelatin implants. Intracanalicular Gelatin Implants in the Treatment of Kerato-Conjunctivitis Sicca, Wallace S. Foulds, British Journal of Ophthalmology (1961) Volume 45 pages 625-627. Foulds discloses that the occlusion of the lacrimal punctum can be performed by inserting a small diameter water soluble gelatin rod into the punctal opening. This gelatin rod is formed from pure powdered gelatin to which a small quantity of distilled waster has been added. The mixture is heated in a water bath until the gelatin dissolves and a thick gel results. By dipping a cold glass rod into the prepared gelatin and withdrawing it, a solid rod of gelatin is formed. The gelatin rod is then inserted into the canaliculus to provide a temporary blockage. After placement this gelatin implant does not last long because the enzymes in the tear fluid quickly broke down the gelatin allowing it to be flushed out of the canaliculus by the tear fluid.

Similarly, U.S. Pat. No. 4,660,546 to Herrick discloses the use of an absorbable material in the shape of a rod as shown in FIG. 2 to temporarily block the canaliculus. Typically, this absorbable material would be a small section of catgut suture. When placed in the canaliculus, the catgut suture swells thereby effectively blocking fluid flow through the canaliculus. The catgut suture remains in place for up to two weeks before it is dissolved by the enzymes in the tear fluid.

Plugs made of silicone elastomer, as shown in FIG. 3, and other more permanent materials which are designed to be retained in the punctal opening are disclosed in several patents, U.S. Pat. No. 3,949,750 to Freeman being an example of these devices. These punctum plugs are generally rod shaped with an oversized tip or barbed portion that dilates and blocks the canaliculus, a smaller neck portion upon which the punctum sphincter ring tightens to hold the plug in place, and a relatively larger head portion which rests upon the top of the punctal opening and prevents the plug from passing down into the canaliculus. Although these plugs are effective and reversible, they tend to become dislodged quite easily. Further, they are somewhat difficult to insert, and occasionally their size and shape can cause tissue damage during insertion or as a result of long term use. In some cases the head of the plug can abrade the scleral tissue of the eye causing irritation. The tissue of the punctum can also be damaged by being dilated by the plugs over extended periods of time.

U.S. Pat. Nos. 5,049,142 and 5,053,030 to Herrick disclose non-absorbable versions of the temporary catgut suture canalicular plug discussed above. However, these plugs were not successful since they did not swell, as did the catgut suture plugs, to fully block flow through the canaliculus. Since these plugs could not swell to fit the canaliculus, they also had a tendency to migrate in the canaliculus and back out into the eye, or to otherwise not remain in place.

Herrick disclosed an improvement to his canalicular plug, as shown in FIG. 4, in U.S. Pat. No. 5,171,270. This improved canalicular plug was in the shape of a rod having a tapered front end, which enabled the plug to be pushed through the punctal opening, and a collapsible flared posterior section. Upon insertion into the canaliculus, the flared section would collapse and conform to the inner dimensions of the canaliculus thereby blocking fluid flow. When this plug was placed in the horizontal canaliculus, a clamping force was developed between the interior wall of the canaliculus and the collapsible flared walls of the plug which held this plug in place. However, at times surgical dissection was required to remove these plugs from the canaliculus.

In U.S. Pat. No. 5,283,063 Freeman discusses plugs made from hydroxyethyl methacrylate (HEMA) hydrogels. The advantage of this material over previous synthetic materials is that the hydrogel in its dry state is stiff which aids insertion. Once placed in the eye it absorbs fluids and becomes soft and flexible, and swells to conform to the canaliculus. However, HEMA does not form a gel but rather a flexible hydrogel similar to a contact lens. Blockage of the canaliculus takes place by deforming the canaliculus around the swollen hydrogel. Freeman further states that the biocompatibility of HEMA has not been satisfactory because proteins are absorbed onto it and denature. This problem is caused by the low water content (40% to 50%) of HEMA which appears to be non-tissue like to protein. Since soft tissue has a water content of at least 80%, a composition that also has at least an 80% water content is required to present a physiological surface to protein.

A cast-in-place plug formed from thermoplastic polymer was disclosed by Schmitt in U.S. Pat. No. 5,469,867. When the polymer is heated slightly above body temperature, it changes to a liquid, flowable state. This allows the polymer to be injected into the canaliculus. When the polymer cools to body temperature it changes back to a solid, non-flowable state. Since the plug is formed in-situ from a liquid polymer, a perfect fit is achieved between the wall of the canaliculus and the solid plug after cooling. There is no flow of fluids around the plug and no migration of the plug. However, since the thermoplastic polymer is reversible, slight warming of the tissues around the canaliculus will cause the plug to change back to a liquid state. This could lead to migration and loss of the liquid polymer forming the plug. Further, oils and fatty acid ester found in tear fluids can dissolve into the polymer and reduce the transition temperature eventually leading to premature loss of the plug.

U.S. Pat. No. 6,234,175 to Zhou, et al., discloses a form-in-place canalicular plug. The plug is formed from a polymer that is rigid when it is frozen and becomes soft and pliable at body temperature. Prior to use, the plug must be stretched to form a thin filament, then held in this configuration until it is frozen to retain this shape. As the thin frozen filament is pushed into the canaliculus through the punctal opening, it warms up and quickly becomes soft. As the filament softens, it shrinks in length and expands to fill the canaliculus until it is met with resistance from the surrounding tissue. During insertion, if the filament is not placed deep enough in the canaliculus, the plug may end up extending through the punctal opening.

Attempts have been made with all of the above plug concepts to reduce the durometer or hardness of the materials forming the plugs. Since these plugs are intended to remain permanently in the patient's punctal opening and/or canaliculus, hard materials, even if flexible, will tend to cause chronic tenderness and pain in the area of the plug.

Therefore, there exists a clear need for a ‘one-size-fits-all’ plug design which would greatly simplify or eliminate the current time-consuming insertion procedures and reduce inventory requirements. A preferred plug is fully contained within the canaliculus in order to prevent the plug from becoming dislodged or does not extend through the punctum, thus abrading eye tissues. Once in place in the canaliculus, the preferred plug material should be soft and pliable, similar to body tissues, to provide maximum long-term comfort to the patient.

### SUMMARY

The present invention is directed to a new, novel, and improved plug for blocking lacrimal fluid flow through the canalicular channels. In a first embodiment this improved canalicular plug is formed from a material which, in its dry state, is stiff enough to be inserted through the punctal opening. In the canaliculus, the material absorbs fluids and swells to form a soft and pliable high water content gel. In alternative embodiments this same material may be partially or fully hydrated prior to insertion.

In the preferred embodiment of the present invention, a rigid or stiff rod shaped, plug is formed from a dry hydrophilic material, the material having a high water content in its hydratedstate. This stiff plug is of a diameter and length such that it can be inserted through the patient's punctum opening and into the canaliculus. This insertion can be accomplished by grasping the stiff plug with forceps and pushing it through the punctum opening. Alternatively, the stiff plug can be placed inside a thin-walled tube which is then placed through the patient's punctum opening. The plug can then be pushed out of the tube using, for example a plunger, and into the canaliculus.

Inside the canaliculus, the plug absorbs lacrimal and other fluids and expands in volume to form a soft and pliable gel conforming to the shape of the caniliculus. A unique characteristic of gels not demonstrated by hydrogels is that gels do not maintain their shape, but rather conform to fit the shape of the canaliculus. This conformation to the shape of the canaliculus restrains the gel from moving and forms an effective blockage to the flow of lacrimal fluid through the canaliculus.

The soft and pliable gel plug that forms remains in the canaliculus unless it is intentionally removed by the physician. To remove it, the physician flushes the gel plug out of the canaliculus and into the nasal cavity utilizing a syringe of physiological solution such as saline. A cannula on the end of the syringe is placed through the punctal opening and the physiological solution is injected into the canaliculus. Because the gel is flexible and does not have a fixede shape the pressure of the solution readily flushes the gel plug through the canaliculus.

In accordance with the invention, gels of water containing natural or synthetic polymeric materials, or composites thereof, can be used. These materials exhibit the special characteristic of forming a rigid or semi-rigid plug in their dry state. Typically, these gels have a high water content which enables them to swell greatly to fill a void in which they are placed without distorting the shape or volume of the void when they absorb water. As a result they provide an improved method and device for occluding the lacrimal drainage system of a patient in order to block the flow of lacrimal fluids through it and more particularly block the flow of fluid through the canaliculus.

In a preferred embodiment the plug is a soft and pliable device which conforms to the geometry of the patient's canaliculus after insertion, blocking the flow of fluids through the canaliculus without deforming the patient's canaliculus and causing pain.

The device can be easily handled and inserted into the canaliculus and does not generally require a range of sizes to achieve an acceptable fit.

Alternatively the device can provide either temporary or permanent blockage of lacrimal fluid flow through the canaliculus and can be easily and safely removed from the canaliculus when desired.

### BRIEF DESCRIPTION OF THE DRAWINGS

The foregoing and other advantages of the present invention will be apparent from the following description of the preferred embodiments of the invention when considered with the accompanying drawings which include the following Figures:

FIG. 1 is a representation of the anatomy of the human eye and associated lacrimal fluid drainage system;

FIG. 2 shows a prior available resorbable plug inserted into the canaliculus in order to temporarily block the flow of lacrimal fluid through the canaliculus;

FIG. 3 shows a prior available silicone elastomer plug inserted into the punctal opening in order to permanently block the flow of lacrimal fluid from the eye through the canaliculus;

FIG. 4 shows a prior available silicone elastomer plug inserted into the canaliculus in order to permanently block the flow of lacrimal fluid through the canaliculus;

FIG. 5 is a first embodiment of the gel plug incorporating features of the present invention in its dry, stiff state;

FIG. 6 shows the dry stiff plug of FIG. 5 being inserted through the punctal opening into the canaliculus;

FIG. 7 shows a partial or fully hydrated plug being inserted through the punctal opening into the canaliculus;

FIG. 8 shows a gel plug in its soft and pliable state after hydration and swelling positioned inside the canaliculus, forming a permanent blockage to the flow of lacrimal fluid from the eye through the canaliculus.

### DETAILED DESCRIPTION OF THE INVENTION

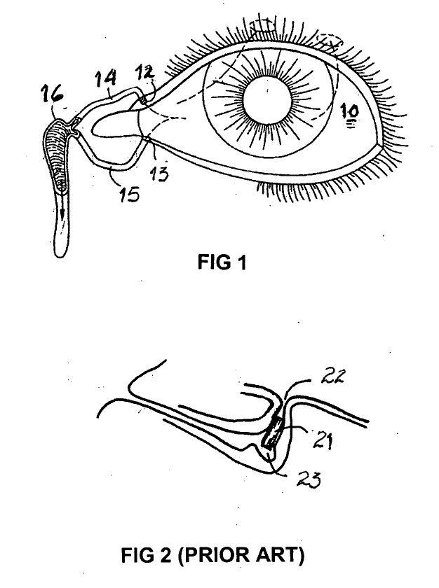

The basic anatomy of the lacrimal fluid drainage system of the human eye 10 is illustrated in FIG. 1. Tears flow into small openings called punctal located in the lids of the eye. Both upper punctum 12 and lower punctum 13 lead to corresponding upper canaliculus 14 and lower canaliculus 15. The upper canaliculus 14 and lower canaliculus 15 merge into the lacrimal sac 16 from which tears travel into the nasal lacrimal duct and drain into the nose. The majority of tears drain through the lower punctum 13 via the canaliculus into the nasal passage. In accordance with features of the invention, a gel plug may be inserted through either punctal into its corresponding canaliculus.

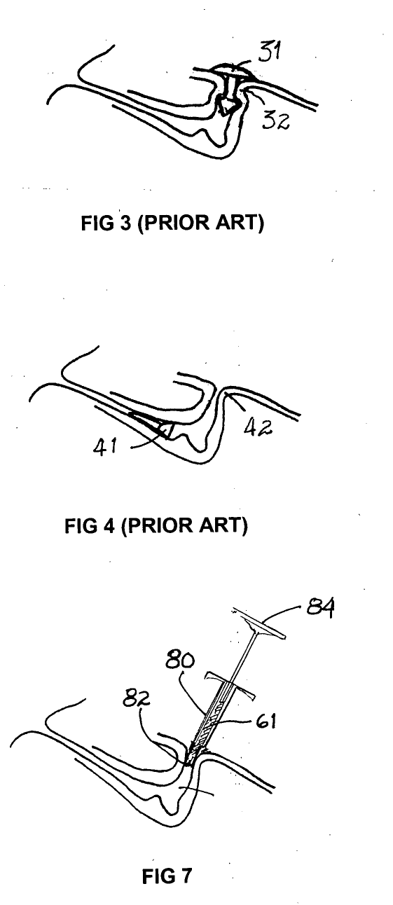

The typical punctal can be opened easily to 0.5 millimeter diameter. However, for insertion of the silicone elastomer punctal plug 31 and canalicular plug 41 shown in FIGS. 3 and 4, the punctal opening is sometimes stretched to 1 millimeter and beyond to accommodate the largest dimensions of these plugs. Each of the punctal openings has a sphincter muscle 32, 42 formed around it, and excessive stretching of the punctal opening can lead to tearing of this muscle. From the punctal openings 12, 13 the upper and lower canaliculus 14, 15 runs vertically for about 2 millimeters and then horizontally for another 8 to 10 millimeters to the lacrimal sac. At its narrowest portion the canaliculus measures about 0.5 millimeter in diameter.

A gel is defined as a material that is sufficiently soft and pliable as to acquire the shape of the container or structure that it is placed in. It is distinguished from a viscous solution in that it does not dissolve in fluids. A gel plug incorporating features of the invention when placed in the canaliculus will conform to the inside walls of the canaliculus without distorting the walls or changing the volume of the void being filled and completely block passage of fluid through the canaliculus.

Soft and pliable gels, which are stiff in their dry state, can be formed from a variety of natural and synthetic materials. In general, as the water content of such hydrophilic materials is increased, the material transitions from a rigid or semi-rigid (stiff) state at or near zero percent water; to a more flexible state at moderate percent water contents; and finally to a soft and pliable gel at high to very high water contents. The transition to a soft and pliable gel typically occurs above about 80% water content.

In contrast, hydrophilic materials, which are usually classified as hydrogels, are composed of polymers that have been modified by cross-linking to covert them from a viscous solution to a gel which swells in the presence of water rather than dissolving. Hydrogel can be made by polymerizing hydrophilic monomers in the presence of crosslinkers or by cross-linking the polymers post polymerization.

Hydrophilic gels can be composed of natural or synthetic polymers. Examples of natural gels, derived from natural polymers, include crosslinked polysaccharides like dextran or crosslinked cellulosic polymers like hydroxypropylmethyl cellulose (HPMC), methyl cellulose (MC) or carboxymethyl cellulose (CMC). An example of a synthetic gels is crosslinked polyvinyl alcohol (PVA).

The following monomers may be used by themselves or in combinations with other monomers in varying amounts to obtain polymers that can swell sufficiently in water to form a gel. These monomers can be either neutral, anionic, or cationic. Examples of the neutral monomers include hydroxyethyl methacrylate (HEMA), glyceryl methacrylate, propyleneglycol methacrylate, polyethyleneglycol methacrylate, acrylamide and its derivatives, polyvinyl alcohol, and hydrolyzed polyacrylonitrile, and N-vinyl pyrrlidinone (NVP). Examples of anionic monomers include acrylic or methacrylic acid, crotonic acid, and styrene sulfonate. Examples of cationic monomers include aminoethyl methacrylate and its derivatives, and vinyl pyridene.

Examples of cross-linkers include ethyleneglycol dimethacrylate (EGDMA), polyethyleneglycol dimethacrylate, and methelene-bis-acrylamide. One can also affect cross-linking by adding small amount of hydrophobic monomers to the polymerizing mixture to create hydrophobic domains that can cause a hydrophilic polymer to form gels in the presence of an aqueous media. At high percent water contents, gels formed from all of these materials become soft, pliable, and tissue like. While hydrophilic materials can be produced to absorb a wide range of fluid concentrations, once formed into a shape that shape is generally fixed and swells in all directions maintaining that shape. Hydrogels when swollen will not assume the shape of the space in which they are placed. In fact swollen hydrogels can be used to dilate the space in which they are placed.

A particularly preferred combination comprises a cross-linked gel prepared from N-vinyl pyrrolidinone (NVP) and a difunctional monomer such as, for example, polyethylene dimethacrylate (PEG200), ethylene dimethacrylate or propylene dimethacrylate and a free radical initiator such as, for example, azo-bis-isobutrylnitrile or Dimethyl-2,2′-azobisisobutyrate. A hydrophobic monomer like methyl methacrylate(MMA), or other esters of methacrylic acid (e.g. ethyl, butyl, or hexyl, etc.) or N-vinyl phthalimide, or styrene or acrylonitrile can be added to aid in stablizing or firming the gel to give it sufficient body to have the gel characteristics without disintegrating when placed in water.

The objective is to produce a cross-linked gel which at hydration equilibrium contains from about 80% to about 97% water. Below 80% the swollen gel will maintain its shape and will not readily deform to take the shape of the space that it is confined within. For canaliculus blocking, it is important to fill the space without deforming and not cause irritation or adverse reactions. At very high water content the gel no longer can be kept together in water and behaves as a viscous fluid which eventually dissolves and dilutes indefinitely.

The gel materials used in accordance with the present invention are preferably biologically inert, biocompatible, and non-immunogenic. No acute physiological activity or response occurs due to the presence of the gel in the canaliculus. Further, the high water content associated with these materials should enable the transport of nutrients and gases to and from the tissues of the canaliculus that the gel is in contact with. This can prevent the eventual denaturation of cells in long term contact with the gel.

FIG. 5 shows a first embodiment of a gel plug 61 in accordance to the present invention. The gel plug 61 in its dry state has a diameter appropriate for insertion through the punctum, generally less than about 1 millimeter and preferably less than about 0.5 millimeter, and a length of less than about 6 millimeters and preferably less than about 3 millimeters. The plug 61 is formed by either casting the material into molds having the desired geometry, by extrusion of rods which are cut to the desired length, or by coring, cutting, or machining bulk pieces of the material into the desired shape. Although other shapes of the dry plug can be made, when the material swells to become a gel, it looses its dry shape and takes on the configuration of the canaliculus where it is placed. The preferred shape is cylindrical for ease of insertion through the punctal opening. The end of the dry plug 52 can be pointed, rounded or made conical to further ease its insertion through the punctum.

FIG. 2 shows a prior available resorbable collagen plug 21 inserted into the canaliculus 23. The plug is made by cutting a twisted catgut suture of 0.3 millimeter to 0.5 millimeter diameter to the desired length of approximately 2 millimeters. The plug 21 is inserted through the punctal opening into the canaliculus 23 using needle-nose forceps. The end of the collagen plug is held by the forceps and pushed through the punctal opening. The tip of the forceps are then used to push the plug into the vertical portion of the canaliculus. The collagen resorbable plug absorbs tear fluid and expands without substantial change in shape, thus fixating the plug 21 in place until it is degraded by the enzymes in the tear fluid.

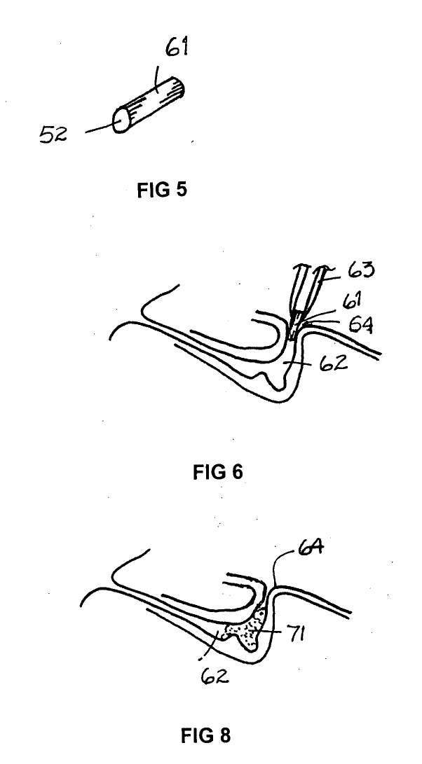

As shown in FIG. 6 a gel plug 61 in its dry state, having dimensions approximately the same as the resorbable collagen plug 21, is inserted into the canaliculus 62 (which is the same as upper canal 14 an lower canal 16 of FIG. 1) in a similar manner to the prior art resorbable collagen plug. When the gel plug is in its dry state, it is rigid or semi-rigid (stiff) depending on the material and the amount of water in the material. The end of the gel plug can be gripped with forceps 63 and pushed through the punctal opening 64 (which is the same as punctum openings 12, 13 of FIG. 1). This is performed in a rapid manner as the gel plug may quickly absorb fluids and swell to become a gel.

Alternatively, as shown in FIG. 7, the gel plug 61 in its dry state can be placed inside a thin-walled tube 80 such as those formed of polyimide plastic having a wall thickness of approximately 0.02 millimeter. The tip 82 of the tube would be placed through the punctal opening 12, 13. Using a plunger 84, the gel plug 61 is then pushed from the inside of the tube 80 into the canaliculus 62. This method of insertion will allow the tube containing the dry plug to be placed through the punctal open without possible hydration and softening of the gel plug until it is pushed from the tube 80.

Inside the canaliculus 62 the dry gel plug 61 absorbs lacrimal and other fluids and swells to become a gel. A hydrophilic material having an 80% water content would swell in volume by 5 times. This represent an increase in diameter and length of 1.7 times. (the cube root of 5). Similarly a hydrophilic material having a 95% water content would swell in volume by 20 times. This represents an increase in diameter and length of 2.7 times. Such a gel plug measuring 0.5 millimeter in diameter by 3.0 millimeters long in its dry state would swell to be nearly 1.5 millimeters in diameter and 9 millimeters long. As illustrated in FIG. 8, this swollen gel plug 71 would conform to the inside of the canaliculus 62 after it swells, thereby forming an effective blockage of lacrimal fluid flow from the eye.

A soft and pliable gel plug would remain in the canaliculus 62 permanently, unless it was intentionally removed by the physician. To remove it, the physician flushes the gel plug out of the canaliculus and into the nasal cavity utilizing a syringe of physiological solution such as saline. A cannula on the end of the syringe is placed through the punctual opening and the physiological solution is injected into the canaliculus. The pressure of the solution expands and lubricates the canaliculus causing the gel plug 61 to be flushed through the canaliculus by the pressure of the solution. At very high water contents, gels are some-what fragile. In some cases the gel plug may be fractured by the solution and flushed out of the canaliculus in several pieces.

### EXAMPLES

In order that the present invention may be more fully understood, the following examples and other comparative results are given by way of illustration only and are not intended to be limiting.

### Example 1

Monomers and initiator were mixed, then filtered through a 0.45 micron Nylon filter into polypropylene vials (about 4 ml) and capped under a nitrogen atmosphere. The mixture was cured at 40° C. for one day, then post cured at 80° C. to obtain cylinders of a dry gel about 25 millimeters diameter by 10 millimeters thick. The cylinders were cored with an appropriately sized cannula to produce rods measuring approximately 0.4 millimeter diameter. These rods were cut into 3 millimeter long gel plugs.

This gel formulation following hydration under equilibrium conditions in physiological saline, had a 94% hydration. The dry gel plugs swelled to 16 times their original volume. The linear dimensions of the gel plugs increased 2.5 times to 1 millimeter diameter by 7.5 millimeters long. The resultant hydrated gel plug was extremely soft and pliable.

### Example 2

Monomer mixture from Example 1 was filled into polypropylene molds having multiple cavities measuring approximately 0.4 millimeter diameter by 3 millimeters deep and the molds were cured as above. The gel plug rods were removed from the molds and extracted with a 50/50 mixture of heptane and acetone to remove unreacted monomers, then dried at 50° C. The dried gel plug rods were placed in pouches and sterilized by gamma irradiation.

Toxicological testing showed the above gel plugs to be non-toxic and biocompatible. Gel plugs were inserted into the canaliculi of Beagle dogs and the flow of lacrimal fluids through the canaliculi was effectively blocked. After a period of time, the gel plugs were removed from the canaliculus by flushing the canaliculi with a syringe filled with saline. The gel plugs were successfully removed from all the canaliculi and flow through the canaliculi was resumed.

As an alternative the gel plug 61 may be partially or fully hydrated before insertion into the canaliculus. In such instance the partially or fully hydrated plug is inserted through the punctum using a tubular device such as shown in FIG. 7. The gel plug can be first hydrated and then drawn into the tubular delivery device or hydrated after placement in the tube of the delivery device by immersing the open end of the tubular delivery device, with gel plug therein, in a container holding the hydration liquid. Also it is possible that hydration can be provided in whole or in part after insertion into the canaliculus by also introducing a hydration fluid from an external source.

In describing the invention, reference has been made to preferred embodiments. Those skilled in the art, and familiar with the disclosure of the subject invention, may recognize additions, deletions, modifications, substitutions, and/or changes which will fall within the purview of the invention as designated in the following claims.

1. A device for blocking the flow of fluids through the canaliculus of a patient comprising a plug of a biocompatible, hydrophilic, non-degradable polymer in a cylindrical form having a diameter and length in a dehydrated condition, the diameter of the cylinder sized to allow insertion of the plug through the punctum and into the canaliculus of said patient; said polymer swellable after insertion by fluids absorbed from the canaliculus to become a gel the dimensions of the swollen plug being sufficient to block the flow of fluid through the canaliculus.

2. The device of claim 1, wherein the dry plug is less than about 1 millimeter in diameter and less than about 6 millimeters long.

3. The device of claim 1, wherein the material forms a gel having a water content of greater than about 80%.

4. A method for blocking the flow of lacrimal fluid through the canaliculus of a patient comprising inserting a cylindrical plug comprising a biocompatible, hydrophilic, non-degradable polymer through the punctum and into the canaliculus of said patient, the cylindrical plug having a diameter and length when dry which is suitable for insertion therein, said polymer being capable of absorbing fluids and swelling to become a gel structure with dimensions suitable for blocking the flow of fluid through the canaliculus.

5. The method of claim 4 wherein the cylindrical plug is inserted in a dry form into the canaliculus, the polymer absorbing fluid from the canaliculus to form a gel.

6. The method of claim 4 wherein the cylindrical plug is fully hydrated prior to insertion into the canaliculus.

7. The method of claim 4 wherein the cylindrical plug is partially hydrated prior to insertion into the canaliculus, the polymer absorbing fluid from the canaliculus to become fully hydrated.

8. The device of claim 4, wherein the dry plug is less than about 1 millimeter in diameter and less than about 6 millimeters long.

9. The device of claim 4, wherein the material forms a gel having a water content of greater than about 80%.

10. A device for blocking the flow of fluids through the canaliculus of a patient comprising a biocompatible, hydrophilic, non-degradable, soft and pliable polymer gel plug completely filling the lumin and in contact with the walls of the canaliculus, thereby blocking fluid flow through the canaliculus.

11. The device of claim 10, wherein the gel plug is formed from a hydrogel material having a water content greater than about 80%.

12. The device of claim 10, wherein the gel plug is inserted into the canaliculus in a dry state where it absorbs fluids from the canaliculus to form a swollen gel.

13. The device of claim 10, wherein the cylindrical plug is hydrated prior to insertion into the canaliculus.
